(19) United States
(12) Patent Application Publication
(10) Pub. No.: US 2021/0283243 A1
(43) Pub. Date: Sep. 16, 2021

(54) LICHENASE-COVID-19 BASED VACCINE

(71) Applicant: iBio, Inc., Bryan, TX (US)

(72) Inventor: Sylvain Marcel, College Station, TX (US)

(21) Appl. No.: 17/198,900

(22) Filed: Mar. 11, 2021

Related U.S. Application Data
(60) Provisional application No. 62/988,141, filed on Mar. 11, 2020.

(51) Int. Cl. A61K 39/215; C12N 15/82; C12N 15/62; C12N 9/42; C07K 14/165

(52) U.S. Cl. CPC A61K 39/215; C12N 15/823; C12N 15/62; C07K 2319/91; C07K 14/165; C12Y 302/01073; C12N 9/2448

(57) ABSTRACT

The present invention includes an immunogenic protein, nucleic acid, plant and immunization comprising a fusion protein that has at least 90% amino acid identity to an amino acid sequence of a modified thermostable lichenase (LicKM) polypeptide as set forth in SEQ ID NO:9, wherein the LicKM polypeptide comprises an N-terminus, a C-terminus, and an inner loop region, and wherein a Receptor Binding Domain (RBD) or a Receptor Binding Motif (RBM) of a coronavirus spike protein is positioned at, at least one of, the N-terminus, the C-terminus, or in a loop region of the LicKM polypeptide.

### CROSS-REFERENCE TO RELATED APPLICATIONS

This application claims priority to U.S. Provisional Application Ser. No. 62/988,141, filed Mar. 11, 2020, the entire contents of which are incorporated herein by reference.

### REFERENCE TO A SEQUENCE LISTING

The present application includes a Sequence Listing which has been submitted in ASCII format via EFS-Web and is hereby incorporated by reference in its entirety. Said ASCII copy, created on Mar. 10, 2021, is named IBIO1013.txt and is 31.9 KB in size.

### TECHNICAL FIELD OF THE INVENTION

The present invention relates in general to the field of vaccines, and more particularly, to a COVID-19 vaccine in a thermostable lichenase (LicKM) carrier.

### STATEMENT OF FEDERALLY FUNDED RESEARCH

Not applicable.

### BACKGROUND OF THE INVENTION

Without limiting the scope of the invention, its background is described in connection with vaccines.

Vaccines are a very effective means for preventing and even eliminating infectious diseases. Although there are a number of efficacious vaccines based on full pathogens, development of safer more potent and cost-effective vaccines based on portions of pathogen (subunit vaccines) is important. During the last two decades several approaches to the expression (bacterial, yeast, mammalian cell culture and plant) and delivery (DNA, live virus vectors, purified proteins, plant virus particles) of vaccine antigens have been developed. All these approaches have significant impact on the development and testing of newly developed candidate vaccines. However, there is a need for improving expression and delivery systems to create more efficacious but safer vaccines with fewer side effects. Some of the desired features or future vaccines are (a) to be highly efficacious (stimulates both arms of immune system), (b) to have known and controlled genetic composition, (c) to have time efficiency of the system, (d) to be suitable for expression of both small size peptides and large size polypeptides, (e) to be suitable for expression in different systems (bacteria, yeast, mammalian cell cultures, live virus vectors, DNA vectors, transgenic plants and transient expression vectors), and (f) to be capable of forming structures such as aggregates or virus like particles that are easy to recover and are immunogenic.

What is needed is an immunization that that be developed quickly, has an enhanced immune response, and can be produced rapidly and effectively.

### SUMMARY OF THE INVENTION

In one embodiment, the present invention includes an immunogenic protein comprising: a fusion protein that has at least 90% amino acid identity to an amino acid sequence of a modified thermostable lichenase (LicKM) polypeptide, wherein the LicKM polypeptide comprises an N-terminus, a C-terminus, and an inner loop region, and wherein a Receptor Binding Domain (RBD) or Receptor Binding Motif (RBM) of a coronavirus spike protein is positioned at, at least one of, the N-terminus, the C-terminus, or in a loop region of the LicKM polypeptide. In one aspect, the loop region is defined from amino acid residues 177 to 184 of the amino acid sequence encoded by SEQ ID NO:9. In another aspect, the coronavirus spike protein comprises SEQ ID NO:10 and SEQ ID NO:11. In another aspect, the immunogenic protein comprises a vaccine antigen. In another aspect, the immunogenic protein has SEQ ID NOS: 1, 3, 5, 7, 12 or 13. In another aspect, the immunogenic protein is further modified to include one or more engineered glycosylation sites. In another aspect, the coronavirus is MERS, SARS, or SARS-CoV-2. In another aspect, the modified thermostable lichenase (LicKM) polypeptide is SEQ ID NO:9. In another aspect, the immunogenic protein is combined with an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

In another embodiment, the present invention includes a method of stimulating an immune response in an animal comprising administering to the animal a composition comprising an immunogenic fusion protein that has at least 90% amino acid identity to an amino acid sequence of a modified thermostable lichenase (LicKM) polypeptide as set forth in SEQ ID NO: 9, wherein the LicKM polypeptide comprises an N-terminus, a C-terminus, and an inner loop region, and wherein a Receptor Binding Domain (RBD) or a Receptor Binding Motif (RBM) of a coronavirus spike protein is positioned at, at least, one of the N-terminus, the C-terminus, or in the loop region of the LicKM polypeptide and a pharmaceutically acceptable carrier, medium or adjuvant. In another aspect, the immune response is at least one of: a humoral immune response, a cellular immune response, or an innate immune response. In another aspect, the coronavirus is MERS, SARS, or SARS-CoV-2. In another aspect, the immunogenic fusion protein has SEQ ID NOS: 1, 3, 5, 7, 12 or 13. In one aspect, the adjuvant is selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, AS04, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

In another embodiment, the present invention includes a method for production of a carrier protein in a plant comprising: (a) providing a plant containing an expression cassette having a nucleic acid encoding a an immunogenic fusion protein that has at least 90% amino acid identity to an amino acid sequence of a modified thermostable lichenase (LicKM) polypeptide, wherein the LicKM polypeptide comprises an N-terminus, a C-terminus, and an inner loop region, wherein a Receptor Binding Domain (RBD) or a Receptor Binding Motif (RBM) of a coronavirus spike protein is positioned at, at least, one of an N-terminus, a C-terminus, or in a loop region, and a pharmaceutically acceptable carrier, medium or adjuvant; and (b) growing the plant under conditions in which the nucleic acid is expressed and the immunogenic fusion protein is produced. In one aspect, the method further comprises the step of recovering the immunogenic protein. In one aspect, a promoter is selected from the group consisting of plant constitutive promoters and plant tissue specific promoters. In one aspect, the immunogenic protein is expressed in leaf, root, fruit, tubercle or seed of the plant. In one aspect, a plant is a Nicotiana sp. plant. In one aspect, the coronavirus is MERS, SARS, or SARS-CoV-2. In one aspect, the method further comprising adding an adjuvant is selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, AS04, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

In another embodiment, the present invention includes a nucleic acid encoding a fusion protein comprising: a fusion protein that has at least 90% amino acid identity to an amino acid sequence of a modified thermostable lichenase (LicKM) polypeptide, wherein the LicKM polypeptide comprises an N-terminus, a C-terminus, and an inner loop region, wherein a Receptor Binding Domain (RBD) or a Receptor Binding Motif (RBM) of a coronavirus spike protein is position at, at least, one of the N-terminus, the C-terminus, or in the loop region. In one aspect, the nucleic acid further comprises a promoter for plant cell expression. In another aspect, the nucleic acid further comprises a plant promoter selected from one or more plant constitutive promoters, and one or more plant tissue specific promoters. In another aspect, the fusion protein is expressed in a leaf, root, fruit, tubercle or seed of the plant. In another aspect, the fusion protein is inserted into a recombinant RNA viral vector has a recombinant genomic component of a tobamovirus, an alfalfa mosaic virus, an ilarvirus, a cucumovirus or a closterovirus. In another aspect, a host plant is a dicotyledon or a monocotyledon. In another aspect, the coronavirus is MERS, SARS, or SARS-CoV-2. In another aspect, the nucleic acid encodes the proteins of SEQ ID NOS: 1, 3, 5, 7, 12 or 13.

### BRIEF DESCRIPTION OF THE DRAWINGS

For a more complete understanding of the features and advantages of the present invention, reference is now made to the detailed description of the invention along with the accompanying figures and in which:

FIG. 1A: Schematic representation of the LicKM protein. 1 is the loop structure containing cloning restriction sites BglII and HindIII. A indicates the region upstream of the loop structure. C indicates the region downstream of the loop structure.

FIG. 1B: Schematic design of the construct with SARS-CoV-2 RBD fused to the c-terminus of LicKM. A poly-histidine tag is fused to the N-terminal of LicKM.

FIG. 1C: Schematic design of the construct with SARS-CoV-2 RBD inserted LicKM. A poly-histidine tag is fused to the N-terminal of LicKM.

FIG. 2 is a graph that shows bulk anti-spike IgG measurements over the experimental time course (left). Table of adjuvant combinations (right).

FIGS. 3A and 3B are graphs that show Day 42 IgG1 and IgG2c sub-titers (FIG. 3A) and IgG1/2c ratios (FIG. 3B).

FIGS. 4A and 4B show the evaluation of functional activity within anti-spike titers. ACE-spike interference assay (FIG. 4A) and pseudovirus neutralization assay (FIG. 4B).

FIG. 5 is an SDS PAGE separation of purified IBIO-201 research antigen. HR; heated and reduced. NHNR; not heated and not reduced

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a cytotoxic T lymphocyte (CTL) epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

To identify epitopes potentially effective as immunogenic compounds, predictions of MHC binding alone are useful but often insufficient. The present invention includes methods for specifically identifying the epitopes within antigens most likely to lead to the immune response sought for the specific sources of antigen presenting cells and responder T cells.

The present invention allows for a rapid and easy assay for the identification of those epitopes that are most likely to produce the desired immune response using the patient's own antigen presenting cells and T cell repertoire. The compositions and methods of the present invention are applicable to any protein sequence, allowing the user to identify the epitopes that are capable of binding to MHC and are properly presented to T cells that will respond to the antigen. Accordingly, the invention is not limited to any particular target or medical condition, but instead encompasses and MHC epitope(s) from any useful source.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” refers to a composition that comprises an antigenic molecule wherein the administration of the composition to a subject result in the development in the subject of a humoral and/or a cellular immune response to the antigenic molecule of interest.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “high-mannose” refers to carbohydrate chains or glycans that contain unsubstituted terminal mannose sugars, and typically contain between five and nine mannose residues, often attached to a chitobiose (G1cNAc₂) core. The name abbreviations are indicative of the total number of mannose residues in the structure, and the position on the carbohydrate of attachment, for example, alpha1,6 is attachment of a mannose in an alpha configuration between carbons 1 and 6, while beta 1,4 is a beta attachment between carbons 1 and 4. The skilled artisan will recognize that the carbohydrates may be high mannose, complex or hybrid, as will beknown to those of skill in the art.

Signal sequences for delivering the proteins of the present invention to different cellular compartments and/or export out of the cell are well-known to the skilled artisan, such as those taught in U.S. Pat. No. 10,577,403, relevant sequences incorporated herein by reference.

As used herein, the term “high-mannose” refers to carbohydrate chains or glycans that contain unsubstituted terminal mannose sugars, and typically contain between five and nine mannose residues, often attached to a chitobiose (G1cNAc₂) core. The name abbreviations are indicative of the total number of mannose residues in the structure, and the position on the carbohydrate of attachment, for example, alpha1,6 is attachment of a mannose in an alpha configuration between carbons 1 and 6, while beta 1,4 is a beta attachment between carbons 1 and 4. The skilled artisan will recognize that the carbohydrates may be high mannose, complex or hybrid, as will beknown to those of skill in the art.

As used herein, the term a “coding sequence” or a sequence which “encodes” a selected polypeptide, refers to a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure.

As used herein, the term “nucleic acid” includes, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986), relevant portion incorporated herein by reference. Suitable programs for calculating the percent identity or similarity between sequences are generally known in the art. Thus, in certain embodiments the present invention will use antigens that include 90, 91, 92, 93, 94, 95, 96, 97, 98, 99 and 100% percent sequence identity at the amino acid level from the protein sequences described herein.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

Many suitable expression systems are commercially available, including, for example, the following: Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, relevant portions incorporated herein by reference.

As used herein, the term “subject” refers to any chordates, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “adjuvant” refers to a substance that non-specifically changes or enhances an antigen-specific immune response of an organism to the antigen. Generally, adjuvants are non-toxic, have high purity, are degradable, and are stable. The recombinant adjuvant of the present invention meets all of these requirements; it is non-toxic, highly pure, degradable, and stable. Adjuvants are often included as one component in a vaccine or therapeutic composition that increases the specific immune response to the antigen. However, the present invention includes a novel adjuvant that does not have to be concurrently administered with the antigen to enhance an immune response, e.g., a humoral immune response. Unlike the common principle of action of other immunologic adjuvants, such as: (1) increasing surface area of an antigen to improve the immunogenicity thereof; (2) causing slow-release of the antigen to extend the retention time of the antigen in tissue; or (3) promoting an inflammatory reaction to stimulate active immune response, the present invention targets the B cells directly to enhance the production of antibodies. Non-limiting examples of adjuvants for use with the present invention include at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, TLR9 ligands.

As used herein, the term “effective dose” refers to that amount of a fusion protein between a modified thermostable lichenase (LicKM) carrier and antigen(s) as enhanced vaccine candidate of the invention sufficient to induce immunity, to prevent and/or ameliorate an infection or to reduce at least one symptom of an infection and/or to enhance the efficacy of another dose of a LicKM-Antigen. An effective dose may refer to the amount of LicKM-Antigen sufficient to delay or minimize the onset of an infection. An effective dose may also refer to the amount of LicKM-Antigen that provides a therapeutic benefit in the treatment or management of an infection. Further, an effective dose is the amount with respect to LicKM-Antigen of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “multivalent” refers to LicKM-Antigen that have multiple antigenic proteins against multiple types or strains of infectious agents.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as LicKM-Antigen of the invention or can be administered separately. Either the protein or an expression vector encoding the protein can be administered to produce an immunostimulatory effect.

As used herein, the term “innate immune response stimulator” refers to agents that trigger the innate or non-specific immune response. The innate immune response is a nonspecific defense mechanism is able to act immediately (or within hours) of an antigen's appearance in the body and the response to which is non-specific, that is, it responds to an entire class of agents (such as oligosaccharides, lipopolysaccharides, nucleic acids such as the CpG motif, etc.) and does not generate an adaptive response, that is, they do not cause immune memory to the antigen. Pathogen-associated immune stimulants act through the Complement cascade, Toll-like Receptors, and other membrane bound receptors to trigger phagocytes to directly kill the perceived pathogen via phagocytosis and/or the expression of immune cell stimulating cytokines and chemokines to stimulate both the innate and adaptive immune responses.

As used herein, the term “protective immune response” or “protective response” refers to an immune response mediated by antibodies or effector cells against an infectious agent, which is exhibited by a vertebrate (e.g., a human), which prevents or ameliorates an infection or reduces at least one symptom thereof. The LicKM-Antigen of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates coronavirus infection or reduces at least one symptom thereof.

As used herein, the term “antigenic formulation” or “antigenic composition” refers to a preparation which, when administered to a vertebrate, e.g. a mammal, will induce an immune response.

As used herein, the terms “immunization” or “vaccine” are used interchangeably to refer to a formulation which contains LicKM-Antigen of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of LicKM-Antigen. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

As used herein, the term “endomembrane reticulum” or “endomembrane system” refers to membranes and organelles in eukaryotic cells that modify, package, and transport proteins. The endomembrane reticulum includes the nuclear envelope, the endoplasmic reticulum, the Golgi apparatus, and even lysosomes. As used in the present invention, the endomembrane reticulum is targeted to modify the protein pos-translationally, for example, to add glycosylation to the protein prior to transport outside the cell, or even specific glycosylation, such as oligomannose and/or other short or long-chain carbohydrates. To reach the endomembrane reticulum, the fusion protein of the present invention can include a signal peptide that targets the protein into specific compartments for post-translational modification and also include glycosylation sequences.

The capability of single plant cell to regenerate and give rise to whole plant with all genetic features of the parent and ii) transfer of foreign genes into a plant genome by a plant-infecting bacterium, Agrobacterium tumefaciens (A. tumefaciens) enabled workers in this field to develop new procedures for crop improvement and stable expression of foreign proteins in plants.

In addition to transgenic plants, with the advances made in molecular plant virology, plant viruses have also emerged as promising tools. Plant viruses have features that range from detrimental to potentially beneficial. The substantial crop losses world-wide due to viral infections have prompted the molecular plant virologists to develop genetic systems that allow manipulation of the virus for management of plant diseases. These genetic systems have also led to the use of viruses as tools, since small plus-sense single-stranded RNA viruses that commonly infect higher plants can be used rapidly amplify virus-related RNAs and produce large amounts of protein.

Both transgenic plants and engineered plant viruses have been used in producing foreign proteins in plant. In the early 1990, transgenic plant technology moved to a new arena as a heterologous expression system for antigens from mammalian pathogens. Since then, a variety of medically important antigens have been expressed in transgenic plants, including hepatitis B surface antigen (HBsAg) E. coli heat-labile enterotoxin, rabies virus glycoprotein, and Norwalk virus capsid protein.

A number of inducible promoters that may allow control over the expression of target genes in transgenic plants have been described. Based on their specificity to a particular class of inducers these promoters could be divided into three groups: i) promoters that are induced at different developmental stages (flowering, senescence, etc.) in different organs (roots, flowers, seeds, etc.), ii) promoters that respond to particular environmental signals (heat-shock, nutritional status, pathogen attack or mechanical wounding), iii) promoters that are induced by chemicals of non-plant origin (tetracycline-, glucocorticoid-, ecdysteroid-, copper- and ethanol-inducible promoters). The latter generally utilize non-plant transcription factors that require chemical inducers for activation. Compared to the first two groups of promoters, chemical-inducible systems have much greater potential for a strict temporal and spatial control of the expression of the target gene expression in transgenic plants. Unfortunately, current inducible plant expression systems have some shortcomings, including leaky promoters or commercially unfeasible manufacturing conditions. The nucleic acid further comprises a plant promoter selected from one or more plant constitutive promoters, and one or more plant tissue specific promoters. The fusion protein can be expressed in a leaf, root, fruit, tubercle or seed of the plant. In one example, the fusion protein is inserted into a recombinant RNA viral vector has a recombinant genomic component of a tobamovirus, an alfalfa mosaic virus, an ilarvirus, a cucumovirus or a closterovirus.

An alternative system for the expression of foreign proteins in plants is based on plant virus vectors. Although plant virus vector-based expression systems have a number of advantages (time, efficient engineering and production, level of target protein expression, environmental safety, etc.) compared to that of transgenic plants, they have some limitations as well. For example, the stability and systemic movement of the recombinant virus may be affected by the size of the target gene. Virus-based vectors are probably less applicable in projects that require coordinated expression of multi-subunit proteins, such as antibodies and enzyme complexes.

The present invention provides vectors and methods for expression of foreign sequences (peptides, polypeptides, and RNA) in plants. Specifically, the present invention relates to vectors and methods for activation of silenced or inactive foreign nucleic acid sequence(s) or gene(s) of interest in plant and animal cells for production of peptides, polypeptides, and RNA in such cells. The vectors used for the activation of silenced or inactive sequence(s) are viral vectors.

The activation of silenced or inactive foreign nucleic acid sequence(s) or gene(s) in plant or animal cells is achieved, in trans, by a factor (e.g., a protein or polypeptide) encoded by a nucleic acid sequence located on the viral vector after the cells are infected with the viral vector. In other words, delivery of activator gene via infection with transient gene delivery viral vector into plant or animal cell activates and results in the expression of target sequence(s). Thus, in the present invention, the activation of silenced or inactive foreign nucleic acid sequence(s) or gene(s) in plant or animal cells is transactivation. It is transactivation because the factor(s) are encoded by nucleic acid sequences that are remotely located, i.e., on the viral vectors, and the factor(s) are free to migrate or diffuse through the cell to their sites of action.

The antigenic portion of the coronavirus may be fused with other sequences that facilitate expression, transport across the cell membranes, tissues and/or systemic delivery. See, for example, U.S. Pat. No. 6,051,239 for sequences which can be fused to the target gene of interest. As part of creating the first component of the transactivation system, a nucleic acid construct is introduced into the plant cell or a plant via a genetic transformation procedure. The nucleic acid construct can be a circular construct such as a plasmid construct or a phagemid construct or cosmid vector or a linear nucleic acid construct including, but not limited to, PCR products. Regardless of the form, the nucleic acid construct introduced is a cassette (also referred to herein as a transfer cassette or an expression cassette) having elements such as promoter(s) and/or enhancer(s) elements besides target gene(s) or the desired coding sequence, among other things. Expression of the target gene, however, depends on transactivation provided by the second component of the invention described further below.

The transactivation system can be a recombinase-based transactivation system or a transcription factor type (with activation and binding domains) based transactivation system. In the recombinase-based transactivation system, the gene of interest (target gene or TG) is cloned into a transfer cassette (or a transformation plasmid) for integration into the plant genome and stable transformation. The target gene in the transformation plasmid is made non-functional by placing a blocking sequence between the promoter (and other regulatory sequences) for driving the expression of the target gene and the target gene. The resulting transfer cassette (or transgenic DNA) is said to have, among other things, the following structure: promoter-blocking sequence-TG.

Different promoters may be used with the present invention, such as, ubiquitous or constitutive (e.g., Cauliflower Mosaic Virus 35S promoter), or tissue specific promoters (e.g., potato protease inhibitor II (pin2) gene promoter, promoters from a number of nodule genes). A number of such promoters are known in the art. Inducible promoters that specifically respond to certain chemicals (copper etc.,) or heat-shock (HSP) are also contemplated. Numerous tissue specific and inducible promoters have been described from plants. The blocking sequence contains a selectable marker element or any other nucleic acid sequence (referred to herein as stuffer) flanked on each side by a recombinase target site (e.g., “FRT” site) with a defined 5′ to 3′ orientation. The FRT refers to a nucleic acid sequence at which the product of the FLP gene, i.e., FLP recombinase, can catalyze the site-specific recombination.

A selectable marker element or stuffer is generally an open reading frame of a gene or alternatively of a length sufficient enough to prevent readthrough. When a suitable recombinase is provided by the second component of the transactivation system to the cells of the transgenic plant containing the transgenic DNA or expression cassette, the recombinase protein can bind to the two target sites on the transgenic DNA, join its two target sequences together and excise the DNA between them so that the target gene is attached to a promoter and/or an enhancer in operable linkage. The recombinase is provided in cells by a viral vector and the recombinase activates the expression of the target gene in cells where it is otherwise silenced or not usually expressed because of the blocking sequence.

It should be noted that the type of recombinase, which is provided to the plant cells in the present invention, would depend upon the recombination target sites in the transgenic DNA (or more specifically in the targeting cassette). For example, if FRT sites are used, the FLP recombinase is provided in the plant cells. Similarly, where lox sites are used, the Cre recombinase is provided in the plant cells. If the non-identical sites are used, for example both an FRT and a lox site, then both the FLP and Cre are provided in the plant cells.

The recombinases used herein are sequence-specific recombinases. These are enzymes that recognize and bind to a short nucleic acid site or a target sequence and catalyze the recombination events. A number of sequence-specific recombinases and their corresponding target sequences are known in the art. For example, the FLP recombinase protein and its target sequence, FRT, are well-characterized and known to one skilled in the art. Briefly, the FLP is a 48 kDa protein encoded by the plasmid of the yeast, Saccharomyces cerevisiae. The FLP recombinase function is to amplify the copy number of the plasmid in the yeast. The FLP recombinase mediates site-specific recombination between a pair of nucleotide sequences, FLP Recognition Targets (FRT's). The FRT is a site for the 48 kDa FLP recombinase. The FRT site is a three repeated DNA sequences of 13 bp each; two repeats in a direct orientation and one in an inverted to the other two. The repeats are separated by the 8 bp spacer region that determine the orientation of the FRT recombination site. Depending of the orientation of the FRT sites FLP-mediated DNA excision or inversion occurs. FRT and FLP sequences can be either wild type or mutant sequences as long as they retain their ability to interact and catalyze the specific excision. Transposases and integrases and their recognition sequences may also be used.

A transfer cassette system may also be used. A viral replicon (e.g., V-BEC) is placed upstream of a target gene. The viral replicon is a viral nucleic acid sequence that allows for the extrachromosomal replication of a nucleic acid construct in a host cell expressing the appropriate replication factors. The replication factor may be provided by a viral vector or a transgenic plant carrying a replicase transgene. Such transgenic plants are known in the art. See, for example, PCT International Publication, WO 00/46350. The constructs of the present invention containing a viral origin of replication, once transcribed, replicate to a high copy number in cells that express the appropriate replication factors. The transfer cassette may contain more than one target gene each linked to a promoter and other elements. Each of the target genes may be transactivated by factors provided by a specific viral vector in a host cell.

In the transcription factor type (for example with activation and binding domains) based transactivation system, the gene of interest (target gene or TG) is cloned into a transfer cassette (or a transformation plasmid) for integration into the host genome (animal or plant) and stable transformation. The target gene will only be expressed when a suitable transcription factor activity is available. This can happen when a fusion protein containing a DNA-binding domain and an activation domain interacts with certain regulatory sequences cloned into the transfer cassette that is integrated into the host genome.

Viral vectors may also be used to deliver factors for transactivation of inactive or silenced target genes in transgenic host cells or organisms. The viral vectors can be RNA type and do not integrate into host genome and the expression is extrachromosomal (transient or in the cytoplasm). Recombinant plant viruses are used in the case of transgenic plant cells or plants. The use of plant viral vectors for expression of recombinases in plants provides a means to have high levels of gene expression within a short time. The autonomously replicating viruses offer several advantages for use as gene delivery vehicles for transient expression of foreign genes, including their characteristic high levels of multiplication and transient gene expression. The recombinant viral vectors used in the present invention are also capable of infecting a suitable host plant and systemically transcribing or expressing foreign sequences or polypeptides in the host plant. Systemic infection or the ability to spread systemically of a virus is an ability of the virus to spread from cell to cell and from infected areas to uninfected distant areas of the infected plant, and to replicate and express in most of the cells of the plant. Thus, this ability of plant viruses to spread to the rest of the plant and their rapid replication would aid in delivery of factors for transactivation throughout the plant and the consequent large-scale production of polypeptides of interest in a short time.

Therefore, the invention also includes the construction of recombinant viral vectors by manipulating the genomic component of the wild-type viruses. Viruses include RNA containing plant viruses. Although many plant viruses have RNA genomes, it is well known that organization of genetic information differs among groups. Thus, a virus can be a mono-, bi-, tri-partite virus. “Genome” refers to the total genetic material of the virus. “RNA genome” states that as present in virions (virus particles), the genome is in RNA form.

Some of the viruses which meet this requirement, and are therefore suitable, include Alfalfa Mosaic Virus (Al MV), ilarviruses, cucumoviruses such as Cucumber Green Mottle Mosaic virus (CGMMV), closteroviruses or tobamaviruses (tobacco mosaic virus group) such as Tobacco Mosaic virus (TMV), Tobacco Etch Virus (TEV), Cowpea Mosaic virus (CMV), and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). Each of these groups of suitable viruses are well characterized and are well known to the skilled artisans in the field. A number of recombinant viral vectors have been used by those skilled in the art to transiently express various polypeptides in plants. See, for example, U.S. Pat. Nos. 5,316,931 and 6,042,832; and PCT International Publication Nos. WO 00/46350, WO 96/12028 and WO 00/25574, the contents of which are incorporated herein by reference. Thus, the methods already known in the art can be used as a guidance to develop recombinant viral vectors of the present invention to deliver transacting factors.

The recombinant viral vector used in the present invention can be heterologous virus vectors. The heterologous virus vectors as referred to herein are those having a recombinant genomic component of a given class of virus (for example TMV) with a movement protein encoding nucleic acid sequence of the given class of virus but coat protein (either a full-length or truncated but functional) nucleic acid sequence of a different class of virus (for example AIMV) in place of the native coat protein nucleic acid sequence of the given class of virus. Likewise, native movement protein nucleic acid sequence instead of the coat protein sequence is replaced by heterologous (i.e. not native) movement protein from another class of virus. For example, a TMV genomic component having an A1MV coat protein is one such heterologous vector. Similarly, an A1MV genomic component having a TMV coat protein is another such heterologous vector. The vectors are designed such that these vectors, upon infection, are capable of replicating in the host cell and transiently activating genes of interest in transgenic plants. Such vectors are known in the art, for example, as described in PCT International Publication, WO 00/46350.

In accordance with the present invention, the host plants included within the scope of the present invention are all species of higher and lower plants of the Plant Kingdom. Mature plants, seedlings, and seeds are included in the scope of the invention. A mature plant includes a plant at any stage in development beyond the seedling. A seedling is a very young, immature plant in the early stages of development. Specifically, plants that can be used as hosts to produce foreign sequences and polypeptides include and are not limited to Angiosperms, Bryophytes such as Hepaticae (liverworts) and Musci (mosses); Pteridophytes such as ferns, horsetails, and lycopods; Gymnosperms such as conifers, cycads, Ginkgo, and Gnetales; and Algae including Chlorophyceae, Phaeophpyceae, Rhodophyceae, Myxophyceae, Xanthophyceae, and Euglenophyceae.

Host plants used for transactivation of genes can be grown either in vivo and/or in vitro depending on the type of the selected plant and the geographic location. It is important that the selected plant is amenable to cultivation under the appropriate field conditions and/or in vitro conditions. The conditions for the growth of the plants are described in various basic books on botany, Agronomy, Taxonomy and Plant Tissue Culture, and are known to a skilled artisan in these fields.

Among angiosperms, the use of crop and/or crop-related members of the families are particularly contemplated. The plant members used in the present methods also include interspecific and/or intergeneric hybrids, mutagenized and/or genetically engineered plants. These families include and not limited to Leguminosae (Fabaceae) including pea, alfalfa, and soybean; Gramineae (Poaceae) including rice, corn, wheat; Solanaceae particularly of the genus Lycopersicon, particularly the species esculentum (tomato), the genus Solanum, particularly the species tuberosum (potato) and melongena (eggplant), the genus Capsicum, particularly the species annum (pepper), tobacco, and the like; Umbelliferae, particularly of the genera Daucus, particularly the species carota (carrot) and Apium, particularly the species graveolens duke, (celery) and the like; Rutaceae, particularly of the genera Citrus (oranges) and the like; Compositae, particularly the genus Lactuca, and the species sativa (lettuce), and the like and the family Cruciferae, particularly of the genera Brassica and Sinapis. Examples of “vegetative” crop members of the family Brassicaceae include, but are not limited to, digenomic tetraploids such as Brassica juncea (L.) Czern. (mustard), B. carinata Braun (ethopian mustard), and monogenomic diploids such as B. oleracea (L.) (cole crops), B. nigra (L.) Koch (black mustard), B. campestris (L.) (turnip rape) and Raphanus sativus (L.) (radish). Examples of “oil-seed” crop members of the family Brassicaceae include, but are not limited to, B. napus (L.) (rapeseed), B. campestris (L.), B. juncea (L.) Czem. and B. tournifortii and Sinapis alba (L.) (white mustard). Flax plants are also contemplated.

Particularly preferred host plants are those that can be infected by A1MV. For example, it is known in the art that alfalfa mosaic virus has full host range. Other species that are known to be susceptible to the virus are: Abelmoschus esculentus, Ageratum conyzoides, Amaranthus caudatus, Amaranthus retroflexus, Antirrhinum majus, Apium graveolens, Apium graveolens var. rapaceum, Arachis hypogaea, Astragalus glycyphyllos, Beta vulgaris, Brassica campestris ssp. rapa, Calendula officinalis, Capsicum annuum, Capsicumfrutescens, Caryopteris incana, Catharanthus roseus, Celosia argentea, Cheiranthus cheiri, Chenopodium album, Chenopodium amaranticol, Chenopodium murale, Chenopodium quinoa, Cicer arietinum, Cichium endiva, Ciandrum sativum, Crotalaria spectabilis, Cucumis melo, Cucumis sativus, Cucurbita pepo, Cyamopsis tetragonoloba, Daucus carota (var. sativa), Dianthus barbatus, Dianthus caryophyllus, Emilia sagittata, Fagopyrum esculentum, Glycine max, Gomphrena globosa, Helianthus annuus, Lablab purpureus, Lactuca sativa, Lathyrus odatus, Lens culinaris, Linum usitatissimum, Lupinus a/bus, Lycopersicon esculentum, Macroptilium lathyroides, Malva parvifla, Matthiola incana, Medicago hispida, Medicago sativa, Melilotus albus, Nicotiana bigelovii, Nicotiana clevelandii, Nicotiana debneyi, Nicotiana glutinosa, Nicotiana megalosiphon, Nicotiana rustica, Nicotiana sylvestris, Nicotiana tabacum, Ocimum basilicum, Petunia x hybrida, Phaseolus lunatus, Phaseolus vulgaris, Philadelphus, Physalis ffidana, Physalis peruviana, Phytolacca americana, Pisum sativum, Solanum demissum, Solanum melongena, Solanum nigrum, Solanum nodiflum, Solanum rostratum, Solanum tuberosum, Sonchus oleraceus, Spinacia oleracea, Ste/Zaria media, Tetragonia tetragonioides, Trifolium dubium, Trifolium hybridum, Trifolium incarnatum, Trifolium pratense, Trifolium repens, Trifolium subterraneum, Tropaeolum majus, Viburnum opulus, Viciafaba, Vigna radiata, Vigna unguiculata, Vigna unguiculata ssp. sesquipedalis, and Zinnia elegans.

A plant virus vector (Av or A1MV) is engineered to express FLP recombinase. The gene for this protein is cloned under subgenomic promoter for coat protein, movement protein or artificial subgenomic promoter. The target gene is cloned into an agrobacterial vector and introduced into nuclear genome to obtain transgenic plants. The target gene is placed under a strong promoter (ubiquitin, dub35, super). However, the expression is silenced by the introduction of NPT or stuffer sequence flanked by FRT (blocking sequence). Target gene is activated by removing the blocking sequences. There can be more than one target gene in a transfer cassette. The target gene(s) is (are) cloned into an agrobacterial vector and introduced into nuclear genome or chloroplast genome. These transformation procedures are well known in the art. Target gene is placed under strong promoter (ubiquitin, dub35, super). However, the expression is silenced by the introduction NPT or stuffer sequences flanked by recombinase recognition sites (e.g., FRT or lox) between the promoter and the TG. The target gene is activated by removing sequences between the promoter and the TG. There could be more than one target gene. The virus vector capable of expressing recombinase in plant cells and transgenic plants (nuclear or chloroplast) that are so made can readily be used to produce target proteins. Transgenic plants are infected with virus containing gene for recombinase.

Inoculation of plants; sprouts, leaves, roots, or stems is done using infectious RNA transcripts, infectious cDNA clones or pregenerated virus material. See, PCT International Publication, WO 00/46350 for guidance on infectious RNA transcripts and procedures for viral infection. Because the time span for target protein production according to the present invention is short (up to 15 days) the expression may not be affected by the gene silencing machinery within the host.

Vaccines. The present invention contemplates immunization for use in both active and passive immunization embodiments. Immunogenic compositions, proposed to be suitable for use as a vaccine, may be prepared most readily directly from immunogenic immunostimulant modified thermostable lichenase (LicKM) and antigens fused to either the N-terminal, C-terminal or inner loop region, proteins and/or peptides prepared in a manner disclosed herein. Preferably the antigenic material is extensively processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline. Examples of antigens for fusion with the LicKM are coronavirus antigens, such as MERS, SARS, and SARS-CoV-2, including the Receptor Binding Domain (RBD) and the Receptor Binding Motif (RBM) of SARS-CoV-2 spike protein.

The preparation of immunizations (also referred to as vaccines) that contain the immunogenic proteins of the present invention as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

Immunizations may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain 1.0-95% of active ingredient, preferably 25-70%.

The proteins may be formulated into the immunization as neutral or salt forms. Pharmaceutically acceptable salts include the acid addition salts (formed with the free amino groups of the peptide) and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2 minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as U.S. Pat. Nos. 3,791,932; 4,174,384 and 3,949,064, as illustrative of these types of assays.

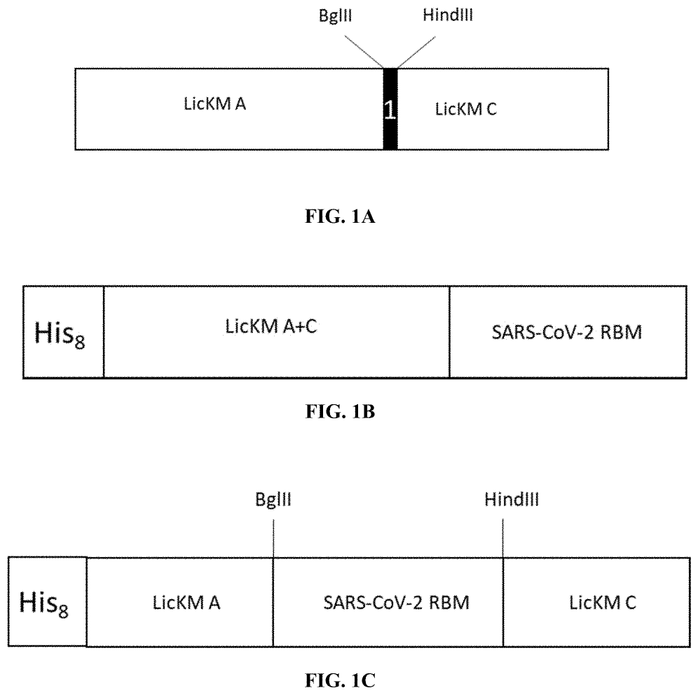

The present invention uses as the immunostimulant a modified thermostable lichenase LicKM carrier to design COVID-19 vaccine candidates. The lichenase LicKM carrier is described in U.S. Pat. Nos. 8,173,408 B2 and 8,591,909 B2, relevant portions incorporated herein by reference. The coronavirus SARS-CoV-2 sequences for the receptor binding motif (RBM) and receptor binding domain (RBD) were fused to the C-terminal end of LicKM (SEQ ID NO:1 and SEQ ID NO:3), respectively). The coronavirus SARS-CoV-2 sequences for the receptor binding motif (RBM) and receptor binding domain (RBD) were fused to the inner loop of LicKM (SEQ ID NO:5 and SEQ ID NO:7). A schematic representation is provided in FIGS. 1A to 1C. SARS-CoV-2 is the coronavirus agent that causes the COVID-19 disease. Lichenase and lichenase modified proteins from Clostridium thermocellum have been used as antigen carrier to enhance antigen stability and increase immune response for several vaccine designs¹⁻⁵. The fusion proteins shown include a poly-histidine tag at the N-terminal of the proteins to allow for affinity purification, which is optional. The fusion to LicKM is expected to increase thermal stability of the antigen. The loop sequence is italicized. The insert is underlined.

SEQ ID NO:1. SARS-CoV-2 receptor binding motif (RBM) and receptor binding domain (RBD) were fused to the C-terminal end of LicKM (amino acid sequence).

MHHHHHHHHGGSYPYKSGEYRTKSFFGYGYYEVRMKAAKNVGIVSSFFT
YTGPSDNNPWDEIDIEFLGKDTTKVQFNWYKNGVGGNEYLHNLGFDASQ
DFHTYGFEWRPDYIDFYVDGKKVYRGTRNIPVTPGKIMMNLWPGIGVDE
WLGRYDGRTPLQAEYEYVKYYPNGRSEFKLVVNTPFVAVFSNFDSSQWE
KADWANGSVFNCVWKPSQVTFSNGKMILTLDREYNSNNLDSKVGGNYNY
LYRLFRKSNLKPFERDISTEIYQAGSTPCNGVEGFNCYFPLQSYGFQPT
NGVGYQPY

SEQ ID NO:2. SARS-CoV-2 receptor binding motif (RBM) and receptor binding domain (RBD) were fused to the C-terminal end of LicKM (nucleic acid sequence).

ATGCATCACCATCACCACCATCATCATGGCGGCTCTTACCCTTATAAGA
GCGGTGAGTACCGGACCAAGAGCTTCTTTGGTTACGGTTACTACGAGGT
GCGGATGAAGGCTGCTAAGAACGTGGGTATCGTGTCCAGCTTCTTTACC
TACACCGGGCCATCTGATAACAACCCTTGGGATGAGATCGACATCGAGT
TCCTTGGTAAGGACACTACCAAGGTGCAGTTCAACTGGTACAAGAACGG
TGTTGGTGGCAACGAGTACCTTCACAACCTTGGCTTTGATGCCAGCCAG
GATTTCCACACTTACGGTTTTGAATGGCGGCCTGACTACATCGACTTCT
ACGTGGACGGTAAGAAGGTGTACAGGGGCACCAGAAATATCCCTGTGAC
TCCTGGCAAGATCATGATGAACCTTTGGCCTGGTATCGGTGTGGATGAG
TGGCTTGGTAGATACGATGGTAGGACTCCTCTGCAGGCTGAGTACGAGT
ACGTTAAGTACTACCCTAACGGCAGATCTGAATTCAAGCTTGTGGTGAA
TACTCCTTTCGTGGCCGTGTTCAGCAACTTCGATTCTAGCCAGTGGGAG
AAAGCTGATTGGGCTAACGGTTCTGTGTTCAACTGCGTGTGGAAGCCTT
CTCAGGTGACCTTCTCTAACGGCAAGATGATTCTGACCCTGGACCGTGA
GTACAACAGCAACAACCTGGATTCTAAGGTCGGCGGCAACTACAACTAC
CTCTACAGGCTGTTCCGGAAGTCCAACCTTAAGCCTTTCGAGAGGGATA
TCAGCACCGAGATCTATCAGGCTGGTTCTACTCCTTGCAATGGCGTTGA
GGGTTTCAACTGCTACTTCCCGCTTCAGTCTTACGGATTCCAGCCTACT
AATGGTGTGGGCTACCAGCCTTATTAG

SEQ ID NO:3. SARS-CoV-2 receptor binding motif (RBM) and receptor binding domain (RBD) were fused to the C-terminal end of LicKM (amino acid sequence).

MHHHHHHHHGGSYPYKSGEYRTKSFFGYGYYEVRMKAAKNVGIVSSFFT
YTGPSDNNPWDEIDIEFLGKDTTKVQFNWYKNGVGGNEYLHNLGFDASQ
DFHTYGFEWRPDYIDFYVDGKKVYRGTRNIPVTPGKIMMNLWPGIGVDE
WLGRYDGRTPLQAEYEYVKYYPNGRSEFKLVVNTPFVAVFSNFDSSQWE
KADWANGSVFNCVWKPSQVTFSNGKMILTLDREYRVQPTESIVRFPNIT
NLCPFGEVFNATRFASVYAWNRKRISNCVADYSVLYNSASFSTFKCYGV
SPTKLNDLCFTNVYADSFVIRGDEVRQIAPGQTGKIADYNYKLPDDFTG
CVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKK

SEQ ID NO:4. SARS-CoV-2 receptor binding motif (RBM) and receptor binding domain (RBD) were fused to the C-terminal end of LicKM (nucleic acid sequence).

ATGCATCACCATCACCACCATCATCATGGCGGCTCTTACCCTTATAAGA
GCGGTGAGTACCGGACCAAGAGCTTCTTTGGTTACGGTTACTACGAGGT
GCGGATGAAGGCTGCTAAGAACGTGGGTATCGTGTCCAGCTTCTTTACC
TACACCGGGCCATCTGATAACAACCCTTGGGATGAGATCGACATCGAGT
TCCTTGGTAAGGACACTACCAAGGTGCAGTTCAACTGGTACAAGAACGG
TGTTGGTGGCAACGAGTACCTTCACAACCTTGGCTTTGATGCCAGCCAG
GATTTCCACACTTACGGTTTTGAATGGCGGCCTGACTACATCGACTTCT
ACGTGGACGGTAAGAAGGTGTACAGGGGCACCAGAAATATCCCTGTGAC
TCCTGGCAAGATCATGATGAACCTTTGGCCTGGTATCGGTGTGGATGAG
TGGCTTGGTAGATACGATGGTAGGACTCCTCTGCAGGCTGAGTACGAGT
ACGTTAAGTACTACCCTAACGGCAGATCTGAATTCAAGCTTGTGGTGAA
TACTCCTTTCGTGGCCGTGTTCAGCAACTTCGATTCTAGCCAGTGGGAG
AAAGCTGATTGGGCTAACGGTTCTGTGTTCAACTGCGTGTGGAAGCCTT
CTCAGGTGACCTTCTCTAACGGCAAGATGATTCTGACCCTGGACCGTGA
GTATAGGGTTCAGCCTACTGAGTCTATCGTGCGGTTCCCTAACATCACC
AACTTGTGCCCTTTCGGCGAGGTGTTCAATGCTACTAGGTTCGCTTCTG
TGTACGCCTGGAACCGGAAGAGGATTTCTAACTGCGTGGCCGATTACAG
CGTGCTGTACAACTCTGCTAGCTTCAGCACCTTCAAGTGCTACGGTGTG
TCTCCTACCAAGCTGAACGATCTGTGCTTCACCAACGTGTACGCTGACT
CTTTCGTGATCAGGGGTGATGAGGTTAGGCAGATTGCTCCTGGTCAGAC
CGGTAAGATCGCTGACTACAACTACAAGCTGCCTGATGACTTCACCGGT
TGCGTGATCGCTTGGAACTCTAACAACCTGGACTCTAAGGTTGGCGGCA
ATTACAACTACCTCTACCGGCTGTTCCGGAAGTCTAACCTTAAGCCTTT
CGAGCGGGATATCAGCACCGAGATCTATCAGGCTGGTTCTACTCCTTGC
AATGGCGTTGAGGGTTTCAACTGCTACTTCCCGCTTCAGTCTTACGGAT
TCCAGCCTACTAATGGTGTGGGCTACCAGCCTTACAGAGTGGTGGTTTT
GTCTTTCGAGCTTCTGCATGCTCCTGCTACTGTTTGCGGTCCTAAGAAG
TAG

SEQ ID NO:5. SARS-CoV-2 sequences for the receptor binding motif (RBM) and receptor binding domain (RBD) were fused to the inner loop of LicKM (amino acid sequence).

MHHHHHHHHGGSYPYKSGEYRTKSFFGYGYYEVRMKAAKNVGIVSSFFT
YTGPSDNNPWDEIDIEFLGKDTTKVQFNWYKNGVGGNEYLHNLGFDASQ
DFHTYGFEWRPDYIDFYVDGKKVYRGTRNIPVTPGKIMMNLWPGIGVDE
WLGRYDGRTPLQAEYEYVKYYPNGRSNSNNLDSKVGGNYNYLYRLFRKS
NLKPFERDISTEIYQAGSTPCNGVEGFNCYFPLQSYGFQPTNGVGYQPY
FKLVVNTPFVAVFSNFDSSQWEKADWANGSVFNCVWKPSQVTFSNGKMI
LTLDREY

SEQ ID NO:6. SARS-CoV-2 sequences for the receptor binding motif (RBM) and receptor binding domain (RBD) were fused to the inner loop of LicKM (nucleic acid sequence).

ATGCATCACCATCACCACCATCATCATGGCGGCTCTTACCCTTATAAGA
GCGGTGAGTACCGGACCAAGAGCTTCTTTGGTTACGGTTACTACGAGGT
GCGGATGAAGGCTGCTAAGAACGTGGGTATCGTGTCCAGCTTCTTTACC
TACACCGGGCCATCTGATAACAACCCTTGGGATGAGATCGACATCGAGT
TCCTTGGTAAGGACACTACCAAGGTGCAGTTCAACTGGTACAAGAACGG
TGTTGGTGGCAACGAGTACCTTCACAACCTTGGCTTTGATGCCAGCCAG
GATTTCCACACTTACGGTTTTGAATGGCGGCCTGACTACATCGACTTCT
ACGTGGACGGTAAGAAGGTGTACAGGGGCACCAGAAATATCCCTGTGAC
TCCTGGCAAGATCATGATGAACCTTTGGCCTGGTATCGGTGTGGATGAG
TGGCTTGGTAGATACGATGGTAGGACTCCTCTGCAGGCTGAGTACGAGT
ACGTTAAGTACTACCCTAACGGCAGATCTAACAGCAACAACCTGGATTC
TAAGGTTGGCGGCAACTACAACTACCTCTACAGGCTGTTCCGGAAGTCC
AACCTTAAGCCTTTCGAGAGGGATATCAGCACCGAGATCTATCAGGCTG
GTTCTACTCCTTGCAATGGCGTTGAGGGTTTCAACTGCTACTTCCCGCT
TCAGTCTTACGGATTCCAGCCTACTAATGGTGTTGGCTACCAGCCGTAC
TTCAAGCTTGTGGTGAATACCCCTTTCGTGGCCGTGTTCAGCAACTTCG
ATTCTAGCCAGTGGGAGAAAGCTGATTGGGCTAACGGTTCTGTGTTCAA
CTGCGTGTGGAAGCCTTCTCAGGTGACCTTCTCTAACGGCAAGATGATT
CTGACCCTGGACCGTGAGTAT

SEQ ID NO:7. SARS-CoV-2 sequences for the receptor binding motif (RBM) and receptor binding domain (RBD) were fused to the inner loop of LicKM (amino acid sequence).

MHHHHHHHHGGSYPYKSGEYRTKSFFGYGYYEVRMKAAKNVGIVSSFFT
YTGPSDNNPWDEIDIEFLGKDTTKVQFNWYKNGVGGNEYLHNLGFDASQ
DFHTYGFEWRPDYIDFYVDGKKVYRGTRNIPVTPGKIMMNLWPGIGVDE
WLGRYDGRTPLQAEYEYVKYYPNGRSRVQPTESIVRFPNITNLCPFGEV
FNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNC
YFPLQSYGFQPTNGVGYQPYRVVVLSFELLHAPATVCGPKKFKLVVNTP
FVAVFSNFDSSQWEKADWANGSVFNCVWKPSQVTFSNGKMILTLDREY

SEQ ID NO:8. SARS-CoV-2 sequences for the receptor binding motif (RBM) and receptor binding domain (RBD) were fused to the inner loop of LicKM (nucleic acid sequence).

ATGCATCACCATCACCACCATCATCATGGCGGCTCTTACCCTTATAAGA
GCGGTGAGTACCGGACCAAGAGCTTCTTTGGTTACGGTTACTACGAGGT
GCGGATGAAGGCTGCTAAGAACGTGGGTATCGTGTCCAGCTTCTTTACC
TACACCGGGCCATCTGATAACAACCCTTGGGATGAGATCGACATCGAGT
TCCTTGGTAAGGACACTACCAAGGTGCAGTTCAACTGGTACAAGAACGG
TGTTGGTGGCAACGAGTACCTTCACAACCTTGGCTTTGATGCCAGCCAG
GATTTCCACACTTACGGTTTTGAATGGCGGCCTGACTACATCGACTTCT
ACGTGGACGGTAAGAAGGTGTACAGGGGCACCAGAAATATCCCTGTGAC
TCCTGGCAAGATCATGATGAACCTTTGGCCTGGTATCGGTGTGGATGAG
TGGCTTGGTAGATACGATGGTAGGACTCCTCTGCAGGCTGAGTACGAGT
ACGTTAAGTACTACCCTAACGGCAGATCTAGGGTTCAGCCTACTGAGTC
TATTGTGCGGTTCCCGAACATCACCAACTTGTGCCCTTTTGGCGAGGTG
TTCAATGCTACCAGGTTCGCTTCTGTGTACGCCTGGAATCGGAAGCGGA
TTTCTAACTGCGTGGCCGATTACAGCGTGCTGTACAACTCTGCTAGCTT
CAGCACCTTCAAGTGCTACGGTGTGTCTCCTACCAAGCTGAACGATCTG
TGCTTCACCAACGTGTACGCTGACTCTTTCGTGATCAGGGGTGATGAGG
TTAGGCAGATTGCTCCTGGTCAGACCGGTAAGATCGCTGACTACAACTA
CAAGCTGCCTGATGACTTCACCGGTTGCGTGATCGCTTGGAACTCTAAC
AACCTGGACTCTAAGGTTGGCGGCAATTACAACTACCTCTACCGGCTGT
TCCGGAAGTCTAACCTTAAGCCTTTCGAGCGGGATATCAGCACCGAGAT
CTATCAGGCTGGTTCTACTCCTTGCAATGGCGTTGAGGGTTTCAACTGC
TACTTCCCGCTTCAGTCTTACGGATTCCAGCCTACTAATGGTGTGGGCT
ACCAGCCTTACAGAGTGGTGGTTTTGTCTTTCGAGCTTCTGCATGCTCC
TGCTACTGTTTGCGGTCCGAAGAAGTTCAAGCTTGTCGTTAATACCCCT
TTCGTGGCCGTGTTCAGCAACTTCGATTCTAGCCAGTGGGAGAAAGCTG
ATTGGGCTAACGGTTCTGTGTTCAACTGCGTGTGGAAGCCTTCTCAGGT
GACCTTCTCTAACGGCAAGATGATTCTGACCCTGGACCGTGAGTATTAG
ctcgag

SEQ ID NO:9. LicKM (amino acid sequence)

MHHHHHHHHGGSYPYKSGEYRTKSFFGYGYYEVRMKAAKNVGIVSSFFT
YTGPSDNNPWDEIDIEFLGKDTTKVQFNWYKNGVGGNEYLHNLGFDASQ
DFHTYGFEWRPDYIDFYVDGKKVYRGTRNIPVTPGKIMMNLWPGIGVDE
WLGRYDGRTPLQAEYEYVKYYPNGRSEFKLVVNTPFVAVFSNFDSSQWE
KADWANGSVFNCVWKPSQVTFSNGKMILTLDREY

SEQ ID NO:10. SARS-CoV-2 receptor binding motif (RBM) and receptor binding domain (RBD)

NSNNLDSKVGGNYNYLYRLFRKSNLKPFERDISTEIYQAGSTPCNGVEG
FNCYFPLQSYGFQPTNGVGYQPY

SEQ ID NO:11. SARS-CoV-2 RBD amino acids 319-567.

RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKK

FIG. 1A: Schematic representation of the LicKM protein. 1 is the loop structure containing cloning restriction sites BglII and HindIII. A indicates the region upstream of the loop structure. C indicates the region downstream of the loop structure. FIG. 1B: Schematic design of the construct with SARS-CoV-2 RBD fused to the c-terminus of LicKM. A poly-histidine tag is fused to the N-terminal of LicKM. FIG. 1C: Schematic design of the construct with SARS-CoV-2 RBD inserted LicKM. A poly-histidine tag is fused to the N-terminal of LicKM.

The antigen used in preclinical studies is comprised of the lichenase subunit (LicKM) fused to SARS-CoV-2 spike protein RBD, spanning sequences 319-567 (genbank QIC53213.1). The research fusion protein incorporates a HIS tag; the commercial antigen is identical without the HIS tag. The sequences are shown below.

SEQ ID NO: 12, [signal peptide (underlined and italicized), 8-HIS tag (underlined), lichenase sequence (normal font), RBD 319-567 (italicized)],

MGFVLFSQLPSFLLVSTLLLFLVISHSCRAHHHHHHHHGGSYPYKSGEY
RTKSFFGYGYYEVRMKAAKNVGIVSSFFTYTGPSDNNPWDEIDIEFLGK
DTTKVQFNWYKNGVGGNEYLHNLGFDASQDFHTYGFEWRPDYIDFYVDG
KKVYRGTRNIPVTPGKIMMNLWPGIGVDEWLGRYDGRTPLQAEYEYVKY
YPNGRSEFKLVVNTPFVAVFSNFDSSQWEKADWANGSVFNCVWKPSQVT
FSNGKMILTLDREYRVQPTESIVRFPNITNLCPFGEVFNATRFASVYAW
NRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPT
NGVGYQPYRVVVLSFELLHAPATVCGPKK

SEQ ID NO: 13, [signal peptide (underlined and italicized), lichenase sequence (normal font), RBD 319-567 (italicized)]

MGFVLFSQLPSFLLVSTLLLFLVISHSCRAGGSYPYKSGEYRTKSFFGY
GYYEVRMKAAKNVGIVSSFFTYTGPSDNNPWDEIDIEFLGKDTTKVQFN
WYKNGVGGNEYLHNLGFDASQDFHTYGFEWRPDYIDFYVDGKKVYRGTR
NIPVTPGKIMMNLWPGIGVDEWLGRYDGRTPLQAEYEYVKYYPNGRSEF
KLVVNTPFVAVFSNFDSSQWEKADWANGSVFNCVWKPSQVTFSNGKMIL
TLDREYRVQPTESIVRFPNITNLCPFGEVFNATRFASVYAWNRKRISNC
VADYSVLYNSASFSTFKCYGVSPTKLNDLCFTNVYADSFVIRGDEVRQI
APGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPY
RVVVLSFELLHAPATVCGPKK

IBIO-0201 Preclinical Immunization Data

The goal of the immunogenicity studies was to evaluate immune responses stimulated by different formulants and adjuvants in combination with the IBIO-201 research antigen (SEQ ID NO: 12) at a fixed 10 μg dose. Six arms were created; antigen alone (Group 24), antigen plus Alum (Group 25), antigen plus glucopyranosyl lipid adjuvant (GLA) (GLA-Alum) (Group 26), antigen plus 3M-052/Alum (Group 27) (3M-052 is a TLR7/8 agonist and is an imidazoquinoline with a C18 lipid moiety), antigen plus squalene emulsion (SE) (Group 28), antigen plus glucopyranosyl lipid adjuvant GLA emulsion with squalene emulsion (GLA-SE) (Group 29), and antigen plus 3M-052 (Group 30). A desired antigen/adjuvant combination would show robust antibody titers, not be biased toward Th2 immune responses where IgG1>>IgG2c and display the ability to interfere with ACE2-spike protein binding.

Mock immunized animals with each formulant/adjuvant combination, but without antigen, were also included in the experiment. Each mouse received intramuscular injections in each leg muscle at day 0 as well as a boost immunization on Day 14. Each arm had 5 mice per group.

Approximately 50 μL of whole blood was collected from each mouse at Day 21, Day 28, Day 35 and Day 42. The mice were sacrificed at Day 42 and spleens were collected for Elispot and FACS analyses. Anti-Spike ELISAs were established to monitor anti-spike titers. Human convalescent antiserum was used as a positive control; various anti-spike antibodies were also considered, but the human serum was generally used throughout. Assays included bulk anti-spike IgG as well as IgG1 and IgG2c subsets. In addition to titers, sera were evaluated for inhibition of ACE2-spike binding (SARS-CoV-2 Surrogate Virus Neutralization Test (sVNT) Kit (RUO), Genscript, Piscataway, N.J., USA) as well as in a pseudovirus neutralization format (Integral Molecular, Philadelphia, Pa., USA), in accordance with the manufacturer's instructions. Mock immunized animals did not show anti-spike titers (data not shown).

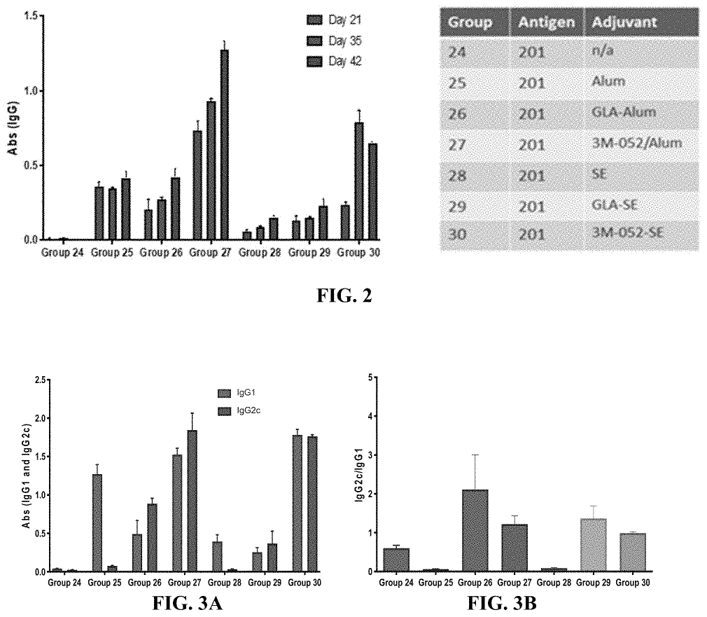

FIG. 2 is a graph that show the bulk anti-spike IgG measurements over the experimental time course (left). Table of adjuvant combinations (right). In FIGS. 3A, 3B, bulk anti-spike titers (pooled from the five mice per group) varied by adjuvant and trended higher over the time course. Both groups 27 and 30 utilized a TLR7/8 agonist adjuvant and showed the strongest anti-spike titers. A stronger immune response may be observed in humans to IBIO-201/TLR7/8 agonist adjuvant combinations.

As shown in FIG. 2, adjuvanted groups showed variable IgG2c/1 ratios. TLR7/8 agonist adjuvanted groups, 27 and 30, showed relatively balanced ratios, while the Alum only (Group 25) showed a strong bias toward IgG1. Strong bias toward IgG1 is common with Alum and is highly suggestive of Th2 skew. Th2 skew has been associated with lung pathology. Generally, an adjuvant combination that promotes Th2 skew would be avoided for this indication.

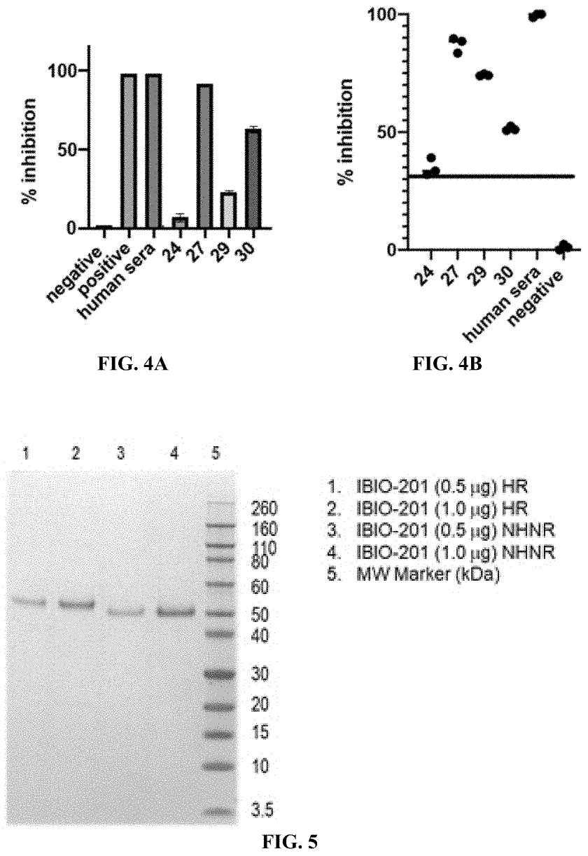

FIGS. 3A and 3B are graphs that show Day 42 IgG1 and IgG2c sub-titers (FIG. 3A) and IgG1/2c ratios (FIG. 3B). As shown in FIGS. 4A, 4B, sera from IBIO-201 immunized mice can functionally interfere with viral protein interactions. In the ACE2 blocking assay (left), sera from 3M-052 adjuvanted groups (27 and 30) interfere with the interaction of purified ACE2 protein and spike. Sera from group 27 (1:10 dilution) show nearly 100% inhibition in this assay format. Likewise, in the pseudovirus neutralization assay using Vero6 cells, sera from IBIO-201 immunized mice strongly neutralized virus infection, comparably to human convalescent serum. In both assays, mouse sera were diluted 1:10. Additional studies are planned to further quantify the neutralization by performing serial dilutions.

FIGS. 4A and 4B show the evaluation of functional activity within anti-spike titers. ACE-spike interference assay (FIG. 4A) and pseudovirus neutralization assay (FIG. 4B).

IBIO-0201 Expression and Purification.

IBIO-201 antigens are expressed via the FASTPHARMING® system using standard approaches. The IBIO-201 research antigen was purified by sequential affinity chromatography and hydrophobic interaction chromatography to very high purity, as shown below.

FIG. 5 is an SDS PAGE separation of purified IBIO-201 research antigen. HR; heated and reduced. NHNR; not heated and not reduced. IBIO-201 purification method development has established robust capture on hydrophobic interaction chromatography using butyl ligand-based resin. Polishing chromatography optimization is ongoing to enhance protein purity to >95%.

An agreed upon battery of release assays will be conducted on IBIO-0201. Assays routinely performed at iBio are listed in the table below.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1—Musiychuck et al., 2007 doi: 10.1111/j.1750-2659.2006.00005.x.
2—Tottey et al., 2018 doi: 10.4269/ajtmh.16-0293
3—Ortega-Berlanga et al., 2016 doi.org/10.1007/s00425-015-2416-z
4—Jones et al., 2015 doi: 10.4161/hv.34366
5—Chichester et al., 2007 doi.org/10.1016/j.vaccine.2007.01.068

What is claimed is:

1. An immunogenic protein comprising:
a fusion protein that has at least 90% amino acid identity to an amino acid sequence of a modified thermostable lichenase (LicKM) polypeptide, wherein the LicKM polypeptide comprises an N-terminus, a C-terminus, and an inner loop region, and wherein a Receptor Binding Domain (RBD) or a Receptor Binding Motif (RBM) of a coronavirus spike protein is positioned at, at least one of, the N-terminus, the C-terminus, or in a loop region of the LicKM polypeptide.

2. The immunogenic protein of claim 1, wherein the loop region is defined by amino acid residues 177 to 184 of the amino acid sequence of SEQ ID NO:9.

3. The immunogenic protein of claim 1, wherein the coronavirus spike protein comprises SEQ ID NO:10 and SEQ ID NO:11.

4. The immunogenic protein of claim 1, wherein the immunogenic protein comprises a vaccine antigen.

5. The immunogenic protein of claim 1, wherein the immunogenic protein has SEQ ID NOS:1, 3, 5, 7, 12 or 13.

6. The immunogenic protein of claim 1, wherein the immunogenic protein is further modified to include one or more engineered glycosylation sites.

7. The immunogenic protein of claim 1, wherein the coronavirus is MERS, SARS, or SARS-CoV-2.

8. The immunogenic protein of claim 1, wherein the modified thermostable lichenase (LicKM) polypeptide is SEQ ID NO:9.

9. The immunogenic protein of claim 1, further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

10. A method of stimulating an immune response in an animal comprising administering to the animal a composition comprising an immunogenic fusion protein that has at least 90% amino acid identity to an amino acid sequence of a modified thermostable lichenase (LicKM) polypeptide as set forth in SEQ ID NO: 9, wherein the LicKM polypeptide comprises an N-terminus, a C-terminus, and an inner loop region, and wherein a Receptor Binding Domain (RBD) or a Receptor Binding Motif (RBM) of a coronavirus spike protein is positioned at, at least, one of the N-terminus, the C-terminus, or in the loop region of the LicKM polypeptide and a pharmaceutically acceptable carrier, medium or adjuvant.

11. The method of claim 10, wherein the immune response is at least one of: a humoral immune response, a cellular immune response, or an innate immune response.

12. The method of claim 10, wherein the coronavirus is MERS, SARS, or SARS-CoV-2.

13. A method for production of a carrier protein in a plant comprising:
(a) providing a plant containing an expression cassette having a nucleic acid encoding a an immunogenic fusion protein that has at least 90% amino acid identity to an amino acid sequence of a modified thermostable lichenase (LicKM) polypeptide, wherein the LicKM polypeptide comprises an N-terminus, a C-terminus, and an inner loop region, wherein a Receptor Binding Domain (RBD) or a Receptor Binding Motif (RBM) of a coronavirus spike protein is positioned at, at least, one of an N-terminus, a C-terminus, or in a loop region, and a pharmaceutically acceptable carrier, medium or adjuvant; and
(b) growing the plant under conditions in which the nucleic acid is expressed and the immunogenic fusion protein is produced.

14. The method of claim 13, further comprising the step of recovering the immunogenic protein.

15. The method of claim 13, wherein a promoter is selected from the group consisting of plant constitutive promoters and plant tissue specific promoters.

16. The method of claim 13, wherein the immunogenic protein is expressed in leaf, root, fruit, tubercle or seed of a plant.

17. The method of claim 13, wherein a plant is a Nicotiana sp. plant.

18. The method of claim 13, wherein the coronavirus is MERS, SARS, or SARS-CoV-2.

19. The method of claim 13, wherein the adjuvant is selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

20. A nucleic acid encoding a fusion protein comprising:
a fusion protein that has at least 90% amino acid identity to an amino acid sequence of a modified thermostable lichenase (LicKM) polypeptide, wherein the LicKM polypeptide comprises an N-terminus, a C-terminus, and an inner loop region, wherein a Receptor Binding Domain (RBD) or a Receptor Binding Motif (RBM) of a coronavirus spike protein is position at, at least, one of the N-terminus, the C-terminus, or in the loop region.

21. The nucleic acid of claim 20, wherein the nucleic acid further comprises a promoter for plant cell expression.

22. The nucleic acid of claim 20, wherein the nucleic acid further comprises a plant promoter selected from one or more plant constitutive promoters, and one or more plant tissue specific promoters.

23. The nucleic acid of claim 20, wherein the fusion protein is expressed in a leaf, root, fruit, tubercle or seed of a plant.

24. The nucleic acid of claim 20, wherein the fusion protein is inserted into a recombinant RNA viral vector has a recombinant genomic component of a tobamovirus, an alfalfa mosaic virus, an ilarvirus, a cucumovirus or a closterovirus.

25. The nucleic acid of claim 20, wherein a host plant is a dicotyledon or a monocotyledon.

26. The nucleic acid of claim 20, wherein the coronavirus is MERS, SARS, or SARS-CoV-2.

27. The nucleic acid of claim 20, wherein the nucleic acid encodes the proteins of SEQ ID NOS: 1, 3, 5, 7, 12 or 13.


SEQUENCE LISTING (13 sequences)

SEQ ID NO: 1 - protein, 302 aa, Artificial Sequence
  Synthetic peptide
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYNSNNLDSKVGGNYNYLYRLFRKSNLKPFERDISTEIYQAGSTPCNGVEGFNCYFPLQSYGFQPTNGVGYQPY

SEQ ID NO: 2 - DNA, 909 bp, Artificial Sequence
  Synthetic primer
atgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtacaacagcaacaacctggattctaaggtcggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgtgggctaccagccttattag

SEQ ID NO: 3 - protein, 441 aa, Artificial Sequence
  Synthetic peptide
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 4 - DNA, 1326 bp, Artificial Sequence
  Synthetic primer
atgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctgaattcaagcttgtggtgaatactcctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtatagggttcagcctactgagtctatcgtgcggttccctaacatcaccaacttgtgccctttcggcgaggtgttcaatgctactaggttcgcttctgtgtacgcctggaaccggaagaggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtcctaagaagtag

SEQ ID NO: 5 - protein, 301 aa, Artificial Sequence
  Synthetic peptide
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSNSNNLDSKVGGNYNYLYRLFRKSNLKPFERDISTEIYQAGSTPCNGVEGFNCYFPLQSYGFQPTNGVGYQPYFKLVVNTPFVAVFSNFDSSQWEKADWANGSVFNCVWKPSQVTFSNGKMILTLDREY

SEQ ID NO: 6 - DNA, 903 bp, Artificial Sequence
  Synthetic primer
atgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctaacagcaacaacctggattctaaggttggcggcaactacaactacctctacaggctgttccggaagtccaaccttaagcctttcgagagggatatcagcaccgagatctatcaggctggttctactccttgcaatggcgttgagggtttcaactgctacttcccgcttcagtcttacggattccagcctactaatggtgttggctaccagccgtacttcaagcttgtggtgaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtat

SEQ ID NO: 7 - protein, 440 aa, Artificial Sequence
  Synthetic peptide
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKFKLVVNTPFVAVFSNFDSSQWEKADWANGSVFNCVWKPSQVTFSNGKMILTLDREY

SEQ ID NO: 8 - DNA, 1329 bp, Artificial Sequence
  Synthetic primer
atgcatcaccatcaccaccatcatcatggcggctcttacccttataagagcggtgagtaccggaccaagagcttctttggttacggttactacgaggtgcggatgaaggctgctaagaacgtgggtatcgtgtccagcttctttacctacaccgggccatctgataacaacccttgggatgagatcgacatcgagttccttggtaaggacactaccaaggtgcagttcaactggtacaagaacggtgttggtggcaacgagtaccttcacaaccttggctttgatgccagccaggatttccacacttacggttttgaatggcggcctgactacatcgacttctacgtggacggtaagaaggtgtacaggggcaccagaaatatccctgtgactcctggcaagatcatgatgaacctttggcctggtatcggtgtggatgagtggcttggtagatacgatggtaggactcctctgcaggctgagtacgagtacgttaagtactaccctaacggcagatctagggttcagcctactgagtctattgtgcggttcccgaacatcaccaacttgtgcccttttggcgaggtgttcaatgctaccaggttcgcttctgtgtacgcctggaatcggaagcggatttctaactgcgtggccgattacagcgtgctgtacaactctgctagcttcagcaccttcaagtgctacggtgtgtctcctaccaagctgaacgatctgtgcttcaccaacgtgtacgctgactctttcgtgatcaggggtgatgaggttaggcagattgctcctggtcagaccggtaagatcgctgactacaactacaagctgcctgatgacttcaccggttgcgtgatcgcttggaactctaacaacctggactctaaggttggcggcaattacaactacctctaccggctgttccggaagtctaaccttaagcctttcgagcgggatatcagcaccgagatctatcaggctggttctactccttgcaatggcgttgagggtttcaactgctacttcccgcttcagtcttacggattccagcctactaatggtgtgggctaccagccttacagagtggtggttttgtctttcgagcttctgcatgctcctgctactgtttgcggtccgaagaagttcaagcttgtcgttaatacccctttcgtggccgtgttcagcaacttcgattctagccagtgggagaaagctgattgggctaacggttctgtgttcaactgcgtgtggaagccttctcaggtgaccttctctaacggcaagatgattctgaccctggaccgtgagtattagctcgag

SEQ ID NO: 9 - protein, 230 aa, Artificial Sequence
  Synthetic peptide
MHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREY

SEQ ID NO: 10 - protein, 72 aa, Artificial Sequence
  Synthetic peptide
NSNNLDSKVGGNYNYLYRLFRKSNLKPFERDISTEIYQAGSTPCNGVEGFNCYFPLQSYGFQPTNGVGYQPY

SEQ ID NO: 11 - protein, 211 aa, Artificial Sequence
  Synthetic peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 12 - protein, 470 aa, Artificial Sequence
  Synthetic peptide
MGFVLFSQLPSFLLVSTLLLFLVISHSCRAHHHHHHHHGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 13 - protein, 462 aa, Artificial Sequence
  Synthetic peptide
MGFVLFSQLPSFLLVSTLLLFLVISHSCRAGGSYPYKSGEYRTKSFFGYGYYEVRMKAAKNVGIVSSFFTYTGPSDNNPWDEIDIEFLGKDTTKVQFNWYKNGVGGNEYLHNLGFDASQDFHTYGFEWRPDYIDFYVDGKKVYRGTRNIPVTPGKIMMNLWPGIGVDEWLGRYDGRTPLQAEYEYVKYYPNGRSEFKLVVNTPFVAVFSNFDSSQWEKADWANGSVFNCVWKPSQVTFSNGKMILTLDREYRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK